(19) United States
(12) Patent Application Publication
(10) Pub. No.: US 2006/0142705 A1
(43) Pub. Date: Jun. 29, 2006

(54) Implantable medication delivery device having needle receiving slot

(76) Inventor: Edgardo Castro Halili, Santa Clarita, CA (US)

Correspondence Address: ARTHUR FREILICH, 9045 CORBIN AVE, #260, NORTHRIDGE, CA, 91324-3343, US

(21) Appl. No.: 11/255,686

(22) Filed: Oct. 20, 2005

Related U.S. Application Data
(63) Continuation of application No. PCT/US04/12464, filed on Apr. 22, 2004.
(60) Provisional application No. 60/465,604, filed on Apr. 24, 2003.

(51) Int. Cl. A61M 31/00

(52) U.S. Cl. 604/288.01

(57) ABSTRACT

A medication delivery device having a housing including at least one needle receiving port comprising a septum and a needle entrance opening. The entrance opening in accordance with the invention comprises an elongate slot having a length L and a width W where L is significantly greater than W and where W is dimensioned to pass only needles smaller than a certain size.

### RELATED APPLICATIONS

This application is a continuation of international application PCT/US04/12464 filed on 22 Apr. 2004 which claims priority based on U.S. provisional application 60/465,604 filed on 24 Apr. 2003. This application claims priority based on said aforementioned applications.

### FIELD OF THE INVENTION

This invention relates generally to implantable medication delivery devices, sometimes referred to as infusion pumps, having one or more inlet ports configured to exchange fluid with a hypodermic needle.

### BACKGROUND OF THE INVENTION

Typical implantable medication delivery devices include two needle receiving ports; i.e., (1) a reservoir fill port and (2) a catheter access port. The reservoir fill port includes an entrance opening configured to allow a hypodermic needle to penetrate a self healing septum for discharging medication to fill an internal reservoir. The catheter access port includes an entrance opening configured to allow a hypodermic needle to penetrate a self healing septum for introducing fluid into or extracting fluid from a catheter. It is often desirable to use differently sized needles for different functions. For example, a larger needle (e.g., 22 gauge) is generally desired for rapid reservoir filling and a smaller needle (e.g., 25 gauge) is generally desired for catheter access.

In order to prevent the inadvertent use of a larger needle in the catheter access port, U.S. Pat. No. 5,328,465 teaches using a screen member above the catheter access port septum for limiting access to hypodermic needles smaller than a predetermined size; e.g., 25 gauge.

U.S. Pat. No. 6,293,922 cites the aforementioned U.S. Pat. No. 5,328,465 and observes that “a few shortcomings of such screens are that they tend to damage hypodermic needles when a needle is inserted not aligned with a hole in the screen”. U.S. Pat. No. 6,293,922 then describes an alternative device intended to eliminate or reduce the possibility of “inadvertent injections of drug directly into the catheter access port without damaging hypodermic needles”. More particularly, U.S. Pat. No. 6,293,922 describes a device wherein a conical depression guides a hypodermic needle into a hole at the center of the depression. The diameter of the hole is chosen to prevent needles having a diameter greater than a certain size, e.g., 25 gauge, from passing therethrough to the septum.

### SUMMARY OF THE INVENTION

The present invention is directed to a medication delivery device having a housing including at least one needle receiving port comprising a septum and a needle entrance opening. The entrance opening in accordance with the invention comprises an elongate slot having a length L and a width W where L is significantly greater than W and where W is dimensioned to pass only needles smaller than a certain size.

The use of an entrance opening slot, rather than a single hole as in U.S. Pat. No. 6,293,922, allows the needle to successively penetrate the underlying septum at randomly located points. This avoids repeated penetrations at the same location and thus enhances the long term reliability of the septum and thus, the device.

Moreover, the use of a slot, rather than a hole, reduces the likelihood of physical damage to the hypodermic needle, e.g., bending. That is, the needle is less constrained when inserted through a slot since only the slot side walls (i.e., slot walls spaced by the width) are likely to engage the needle. The slot end walls spaced by the slot length provide significant freedom of movement in the length direction and reduce the potential of needle damage.

In an exemplary embodiment of the invention, the entrance opening of a catheter access port comprises a slot dimensioned to admit 25 gauge needles (having an outer diameter of about 0.0205 inches), or smaller. In a preferred embodiment, the access port entrance opening slot has a length L, of about 0.1200 inches, and a much smaller slot width W of about 0.0210 inches to prevent needles larger than 25 gauge from passing therethrough.

The slot is preferably located at the bottom of an oval shaped depression whose sloping peripheral wall surface converges downwardly toward the slot. The peripheral wall surface is preferably smooth to enable it to readily guide the needle tip toward the slot. The peripheral wall flares upwardly and outwardly from the slot to provide a large target area for receiving and guiding the needle tip.

Slotted entrance openings in accordance with the invention are useful in a variety of differently configured implantable medication delivery devices. For example, in one application, multiple devices each having only one needle receiving port are configured with differently sized entrance slots so that each device will reject needles larger than a certain size. This facilitates the use of a plurality of such devices implanted in close proximity in a body since it reduces the risk of introducing a wrong needle into a port.

Additionally, the use of differently sized entrance slots facilitates the inclusion of multiple medication reservoirs within a single device. That is, it is convenient to provide an implantable device having two or more reservoirs with each reservoir having a separate fill port and with the multiple fill ports each having a differently sized slot. This feature reduces the risk of a particular needle being introduced into the wrong fill port.

### BRIEF DESCRIPTION OF THE FIGURES

FIG. 1 is a plan view of an exemplary implantable medication delivery device in accordance with the invention having two needle receiving ports;

FIG. 2 is a schematic diagram illustrating the functional components of the delivery device of FIG. 1;

FIG. 3 is a plan view of a preferred inlet port in accordance with the present invention;

FIG. 4 is a sectional view taken substantially along the plane 4-4 of FIG. 3;

FIG. 5 is a sectional view taken substantially along the plane 5-5 of FIG. 3;

FIG. 6 is plan view of an exemplary single port implantable device in accordance with the invention;

FIG. 7 is a schematic diagram illustrating the functional components of the delivery device of FIG. 6;

FIG. 8 is a plan view of an alternative device in accordance with the invention having two needle receiving ports;

FIGS. 9 and 10 are plan views of alternative implantable devices in accordance with the invention having three needle receiving ports;

FIG. 11 is a schematic diagram illustrating the functional components of the devices of FIGS. 9 and 10; and

FIGS. 12 and 13 are plan views illustrating alternative slotted needle entrance openings in accordance with the present invention.

### DETAILED DESCRIPTION

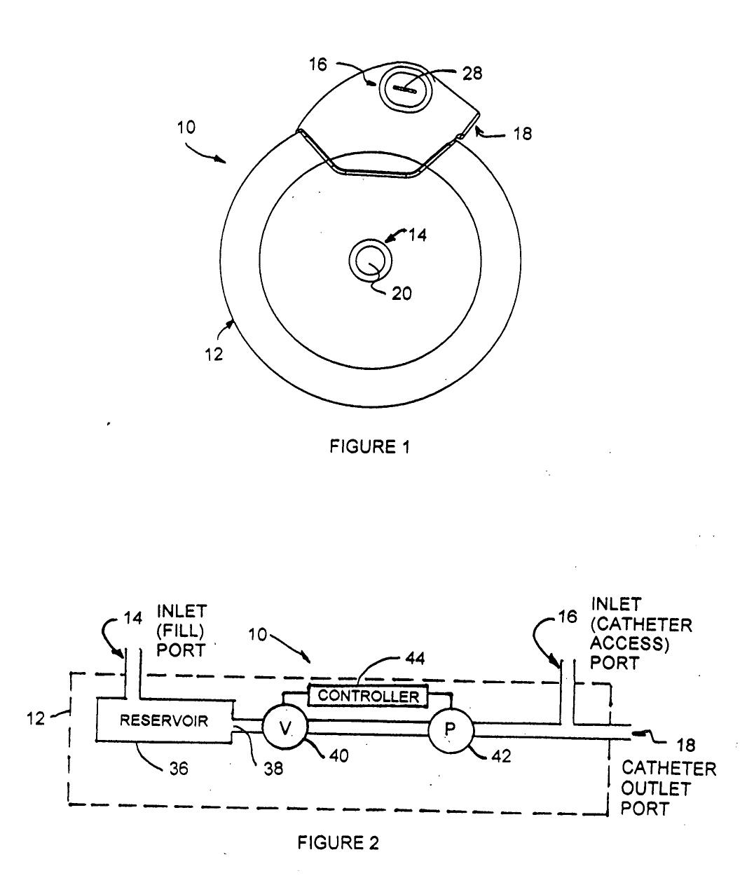

Attention is initially directed to FIG. 1 which schematically illustrates an exemplary implantable medication delivery device 10 in accordance with the invention. The device 10 comprises a housing 12 enclosing an interior volume and defining first and second needle receiving inlet ports 14 and 16. Inlet port 14 is used for filling an internal medication reservoir and inlet port 16 is used for accessing a catheter outlet port 18 to either extract a sample and/or introduce a fluid.

The inlet port 14 includes a conventional needle receiving entrance opening 20 which is shown as being large enough to accept virtually any size hypodermic needle. The inlet port 16 includes a needle receiving entrance opening 28 comprising a slot having a width narrow enough to prevent the entry of needles larger than a certain size.

FIG. 2 schematically illustrates the internal and functional aspects of the medication delivery device 10. More particularly, note inlet port 14 which opens through the wall of housing 12 into a reservoir 36 mounted in the housing interior volume. The reservoir outlet 38 is coupled via a flow path comprising a flow metering mechanism including, for example, a valve mechanism 40 and a pump mechanism 42, to the catheter outlet port 18. The valve mechanism 40 and pump mechanism 42 can be controlled, e.g., by a battery operated controller 44, to extract medication from the reservoir 36 at some predetermined rate for discharge through the catheter outlet port 18.

FIG. 2 also shows the aforementioned port 16 which provides direct access to the catheter outlet port 18 and is useful for either extracting a fluid sample and/or introducing a fluid, e.g., medication or dye. It should be understood that the device as functionally depicted in FIG. 2 is intended to be representative only. Various types of pumping mechanisms and valve mechanisms in various configurations can in fact be used for extracting medication from the reservoir 36 for discharge at a controlled rate through the catheter outlet port 18.

In the use of many existing devices, it is typical to use a large hypodermic needle, e.g., 22 gauge, for rapidly filling the reservoir 36 through the inlet port 14. In contrast, however, for safety reasons, it is generally preferred to use a smaller needle, e.g., 25 gauge, to access the catheter via port 16. In order to reduce the likelihood of a clinician inadvertently using the larger needle in port 16, it has been suggested (e.g. U.S. Pat. No. 6,293,922) that the port 16 have a central needle entrance hole whose diameter is only large enough to accept the smaller needle.

Attention is now directed to FIG. 3 which comprises an enlarged plan view of inlet port 16 in accordance with the present invention. FIG. 3 shows needle entrance opening 28 as comprising an elongate slot 48 having a length L and a width W. In accordance with a preferred embodiment of the invention, in order to prevent needles larger than 25 gauge from passing through the slot 48, the width W, defined by side walls 50 and 52 (FIG. 4), should be only slightly greater than 0.0205. The length L of the slot 48 is defined by end walls 56 and 58 (FIG. 5). The length L in accordance with the invention is at least four times the width W.

The slot 48 is preferably formed in the bottom of an oval depression 62 formed in a frame structure 64. More particularly, note that frame structure 64 defines an oval top frame 66 surrounding an oval region 68. The depression 62 is formed in the region 68 by a downwardly converging peripheral wall surface 70 leading to the slot 48.

The slot 48 overlays a self healing septum 76 which is mounted above chamber 78. Chamber 78 is surrounded by a filter ring 80. A passageway 82 outside of the filter ring 80 communicates with channel 84 which leads to the aforementioned catheter outlet port 18.

The following table shows the dimensions of standard hypodermic needles for various gauge sizes. The table demonstrates that in order to restrict access to slot 48 to needles 25 gauge and smaller, the slot should have a width W of about 0.0210 inches between side walls 50 and 52.

TABLE 1
DIMENSIONS OF NEEDLES

| | | | | | | Canula: |
|---|---|---|---|---|---|---|
| | | | | Canula: | | Inside |
| | | Point: | Canula: | Outside Diameter | | Diameter |
| | Point: | Length | Length | (Inches) | | (Inches) |
| Gage | Style | (Inches) | (Inches) | Maximum | Minimum | (Minimum) |
| 27.00 | A | 0.078 | ¼, ⅜, ½, ⅝, ¾, 1 | 0.0165 | 0.0155 | 0.0075 |
| | C | 0.47 | ¼, ⅜ | .0165 | .0155 | .0075 |
| 26.00 | A | .086 | ¼, ⅜, ½, ⅝, ¾, ⅞, 1, 1¼, 1½ | .0185 | .0175 | .0095 |
| | C | .047 | ¼, ⅜ | .0185 | .0175 | .0095 |
| 25.00 | A | .094 | ¼, ⅜, ½, ⅝, ¾, ⅞, 1, 1¼, 1½, 2 | .0205 | .0195 | .0095 |
| | B | .070 | ¾ | .0205 | .0195 | .0095 |
| 24.00 | A | .102 | ⅜, ½, ⅝, ¾, 1¼, 1½, 2 | .0225 | .0215 | .0115 |
| | B | .070 | ¾ | .0225 | .0215 | .0115 |
| 23.00 | A | .117 | ½, ⅝, ¾, 1, 1¼, 1½, 2 | .0255 | .0245 | .0125 |
| | B | .078 | ¾ | .0255 | .0245 | .0125 |
| 22.00 | A | .133 | 1, 1¼, 1½, 2 | .0285 | .0275 | .0155 |
| | B | .086 | 1, 1¼ | .0285 | .0275 | .0155 |
| 21.00 | A | .148 | 1, 1¼, 1½, 2, 3 | .0325 | .0315 | .0195 |
| | B | .094 | 1¼, 1½ | .0325 | .0315 | .0195 |
| 20.00 | A | .164 | ¾, 1, 1¼, 1½, 1¾, 2, 2½, 3 | .036 | .034 | .022 |
| | B | .109 | ¾, 1, 1¼, 1½ | .036 | .034 | .022 |
| 19.00 | A | .195 | 1, 1¼, 1½, 1¾, 2, 2½, 3 | .0435 | .0405 | .0255 |
| | B | .125 | 2.00 | .0435 | .0405 | .0255 |
| 18.00 | A | .226 | 1, 1¼, 1½, 2, 2½, 3 | .0505 | .0475 | .0135 |
| | B | .148 | 2.00 | .0505 | .0475 | .0135 |
| 17.00 | A | .258 | 1½, 2, 2½, 3, 3½ | .0595 | .0565 | .0365 |
| | B | .172 | 1½, 2 | .0595 | .0565 | .0365 |
| 16.00 | A | .289 | 1½, 2, 2½, 3, 4 | .0665 | .0635 | .0145 |
| | B | .187 | 1½, 2 | .0665 | .0635 | .0145 |
| 15.00 | A | .320 | 1½, 2, 2½, 3, 3½ | .074 | .070 | .048 |
| | B | .203 | 1½, 2 | .074 | .070 | .048 |
| 13.00 | A | .383 | 1½, 2, 3½ | .097 | .093 | .067 |
| | B | .234 | 1½, 2 | .097 | .093 | .067 |

1. Tolerance ∵ 10%
1. Tolerances:
2. ⅜ inch and shorter, ± 1/32 inch
3. ½ inch to ¾ inch, ± 3/64 inch
4. ⅞ inch to 1½ inch, ± 1/16 inch
5. 1¾ inches to 2½ inches, ± 5/64 inch

FIG. 4 depicts a hypodermic needle 88 being inserted into the entrance opening slot 48 of port 16. Note that the needle 88 is shown as being too large to fit through the slot width between side walls 50 and 52 which are assumed to be spaced by a width of 0.0210. Thus, FIG. 4 illustrates how the slot width dimension rejects needles larger than 25 gauge. FIG. 5 represents a 25 gauge needle 90 penetrating the slot 48 and the underlying septum 76 to enter the chamber 78.

The use of a slot 48 having a width W and a length L considerably greater than W, affords multiple advantages as contrasted with the prior art. Initially, because the length L is several times greater than the width W, the needle is likely to pierce the septum at different points during successive penetrations. By not restricting the needle piercing to a single location, the long term reliability and useful life of the septum and device is enhanced. Moreover, the potential of needle damage is reduced because the needle is physically constrained only by the width dimension between side walls 50 and 52 and not by the length dimension between end walls 56 and 58. Further, the use of an oval depression 62 and slotted entrance opening 48 provides a larger target area for a clinician to properly insert the needle through the patient's skin in alignment with the oval depression peripheral surface 70. Note that the peripheral surface 70 flairs upwardly and outwardly from the entrance opening slot 48 to define the large oval depression 62 and target area. The peripheral surface 70 converges downwardly from frame 66 toward slot 48 and is preferably quite smooth so as to be able to easily guide a needle tip toward the slot 48.

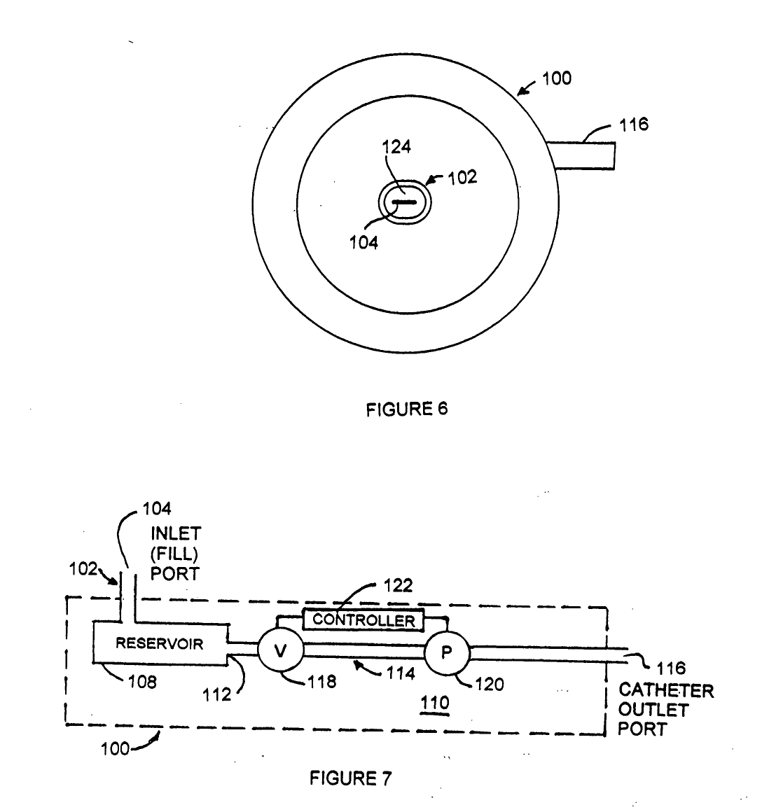

Attention is now directed to FIG. 6 which shows an exemplary implantable device 100 having a single inlet port 102 in accordance with the present invention. That is, the inlet port 102 includes a needle receiving entrance opening 104 comprising a slot of the type shown in FIGS. 3-5. The entrance opening 104 communicates with a reservoir 108 (FIG. 7) mounted in the interior volume 110 of the device 100 housing. The reservoir outlet 112 is coupled by flow metering mechanism 114 to a catheter outlet port 116. The flow metering mechanism 114, as has been previously described, can be comprised of a controllable valve mechanism 118 and a controllable pump mechanism 120. Both the valve mechanism 118 and the pump mechanism 120 are depicted as being controlled by a battery operated controller 122.

In accordance with the invention, the entrance opening slot 104 has a width W and a length L at least four times greater than W. The slot 104, as has been previously described, is surrounded by a slopping peripheral wall surface 124 which converges downwardly toward the slot. The slot width W is selected to prevent penetration by a hypodermic needle larger than a certain size. It is contemplated in accordance with the invention that a patient can have multiple single port devices 100 implanted in his body in close proximity. The devices can be used to discharge needed flow rates of different medications or of the same medication to different sites. Regardless, in order to reduce the risk of penetrating a port 102 with a wrong needle, the multiple devices are selected so as to have different slot widths.

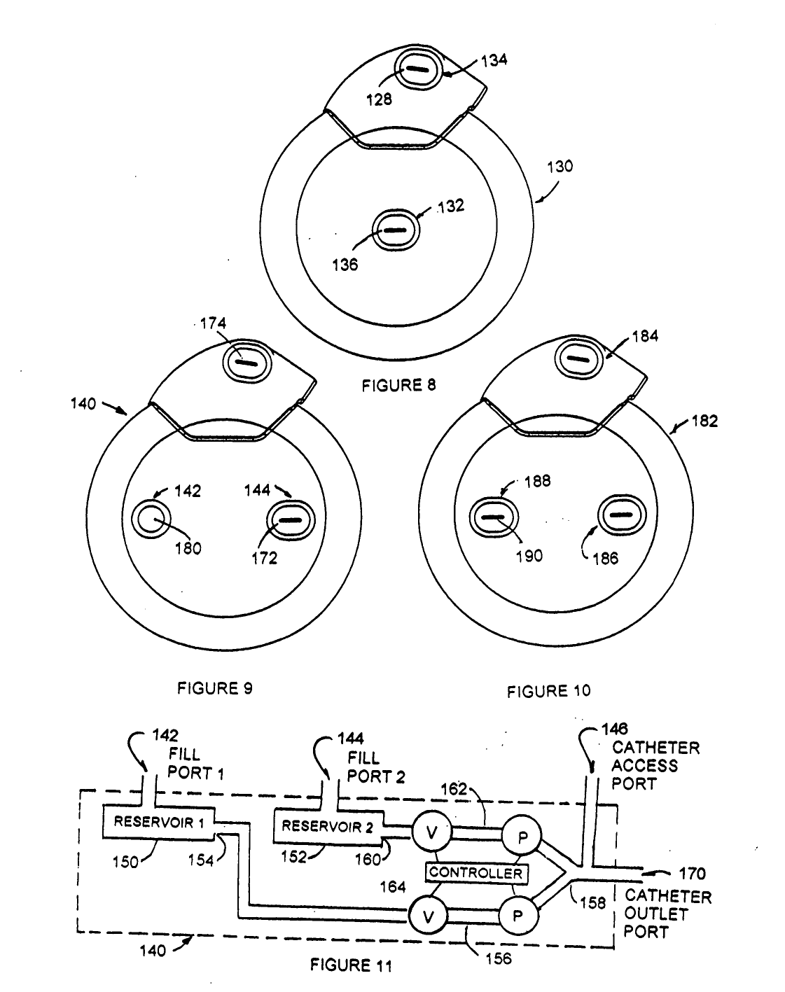

FIG. 8 is a plan view of a further alternative configuration of the invention. More particularly, FIG. 8 depicts an implantable device 130 having a first needle receiving port 132 and a second needle receiving port 134. The ports 132 and 134 respectively have slotted entrance openings 136 and 138 having different widths W. The port 132 corresponds to port 14 of FIG. 1 and functions to fill a reservoir mounted in the device 130. The port 134 corresponds to port 16 of FIG. 1 and is used for catheter access. In a preferred embodiment, port 132 slot 136 has a width of at least 0.0285 inches for accepting 22 gauge needles and smaller and port 134 slot 138 has a width of at least 0.0205 inches for accepting 25 gauge needles and smaller.

The implantable devices thus far described with reference to FIGS. 1, 6, and 8 contemplate the inclusion of a single reservoir within the device housing. It is contemplated, however, that in accordance with the invention a single device housing can include two or more medication reservoirs with each reservoir having a separate inlet port. The utilization of an implantable device with multiple medication reservoirs allows for the administration of more complex and precise therapeutic procedures.

FIG. 9 depicts an implantable device 140 having three needle receiving ports, i.e., 142, 144, and 146. Port 146 is shown as comprising a catheter access port and is analogous to the port 16 previously described in connection with FIG. 1. Ports 142 and 144 are coupled to separate reservoirs mounted within the interior volume of device 140. That is, port 142 functions as the inlet to the first reservoir 150 (FIG. 11) and port 144 functions as the inlet to the second reservoir 152. FIG. 11 shows the outlet 154 of reservoir 150 coupled through a flow metering mechanism 156 to a Y-connector 158. The outlet 160 of reservoir 152 is similarly shown coupled to a flow metering mechanism 162 whose output is coupled to the Y-connector 158. The flow metering mechanisms 156 and 162 can be controlled by separate controllers or by a common battery operated controller 164. FIG. 11 depicts the flow metering mechanisms 156 and 162 as converging at the Y-connector 158 to contribute respective flows to the path leading to catheter outlet port 170. FIG. 11 also depicts catheter access port 146 coupled between the Y-connector 158 and the outlet 170.

In the device of FIG. 9, port 144 is shown as having a slotted entrance opening 172. Catheter access port 146 is shown as having a slotted entrance opening 174. In accordance with the present invention, it is contemplated that the respective slotted entrance openings will have different widths W to reduce the risk of the wrong needle being inserted into a port. FIG. 9 depicts the port 142 as defining a large entrance opening 180, analogous to the port 14 of FIG. 1, for accommodating hypodermic needles of virtually any size.

FIG. 10 illustrates a further device configuration 182 wherein all of the needle receiving ports 184, 186, and 188 have slotted entrance openings. Thus, FIG. 10 differs from FIG. 9 in that port 188 has a slotted opening 190 of a defined width W in contrast to the large entrance opening 180 of port 142 in FIG. 9. The utilization of multiple ports, as shown in FIG. 10, with each having a differently defined slot width further reduces the risk of wrong needle insertion. As should be apparent, the device 182 of FIG. 10 can be used with the reservoir and flow metering system shown in FIG. 11.

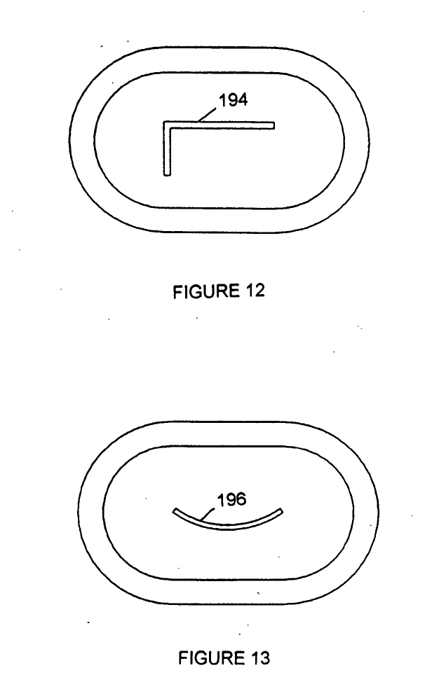

Although the figures mentioned thus far all depict an entrance opening in the form of a straight elongate slot, it is recognized that the entrance opening slots can be alternatively configured. Thus, FIG. 12 illustrates one such alternative in which the elongate slot 194 is comprised of two legs. FIG. 13 illustrates a further alternative configuration in which the elongate slot 196 is curved. Other slot configurations can also be used.

From the foregoing, it should be recognized that improved needle receiving port configurations have been disclosed for implantable medical delivery devices. Although only a limited number of inlet port configurations have been specifically disclosed, it should be understood that various alternative and equivalent configurations will occur to those skilled in the art which are consistent with the teachings of the present invention and within the intended scope of the appended claims.

1. An implantable medication delivery device comprising:
a housing defining an interior volume;
a needle receiving port in said housing for communicating with said interior volume;
said port comprising a septum and an entrance opening overlaying said septum;
said entrance opening comprising a slot having a length L and a width W where W is selected to prevent penetration by a needle larger than a certain size and where L is greater than four times W.

2. The device of claim 1 wherein said needle receiving port defines an oval shaped depression having a sloping peripheral wall converging downwardly toward said entrance opening.

3. The device of claim 1 including a reservoir mounted in said interior volume; and wherein
said needle receiving port communicates with said reservoir for supplying medication thereto

4. The device of claim 1 further including an outlet port in said housing; and wherein
said needle receiving port communicates with said outlet port.

5. An implantable medication delivery device comprising:
a housing defining an interior volume;
first and second reservoirs mounted in said interior volume;
said housing defining first and second needle receiving ports respectively coupled to said first and second reservoirs, each of said ports comprising a septum and an entrance opening overlaying said septum; and wherein
at least one of said entrance openings comprises a slot having a length L and a width W where W is selected to prevent a needle larger than a certain size from penetrating the underlying septum.

6. The device of claim 5 wherein said needle receiving port having an entrance opening slot further includes:
an oval shaped depression having a sloping peripheral wall converging downwardly toward said slot.

7. The device of claim 5 further including an outlet port in said housing; and
a metering flow path coupling each of said reservoirs to said outlet port.

8. An implantable medication delivery device comprising:
a medication reservoir;
an outlet port adapted for coupling to a catheter;
a flow path coupling said reservoir to said outlet port;
a first needle receiving port for receiving medication to fill said reservoir;
said first port comprising a septum and a first needle entrance opening overlaying said septum;
a second needle receiving port for extracting fluid from or introducing fluid to said outlet port;
said second port comprising a septum and a second needle entrance opening overlaying said septum; and wherein
at least one of said first and second needle entrance openings comprises a slot having a length L and a width W where L is greater than four times W.

9. The device of claim 8 wherein said second entrance opening comprises a slot having a width W greater than 0.0205 inches for passing a 25 gauge needle and less than 0.0275 inches for rejecting a 22 gauge needle.

10. The device of claim 9 wherein said second inlet port further defines an oval shaped depression having a sloping peripheral wall converging downwardly toward said second opening slot.

11. The device of claim 8 wherein said first entrance opening is large enough to pass a 22 gauge needle.

12. An implantable medication delivery device comprising:
a medication reservoir;
a catheter outlet port;
a flow path coupling said reservoir to said catheter outlet port;
a first inlet port coupled to said reservoir;
said first inlet port comprising a septum and a first needle entrance opening overlaying said septum;
a second inlet port coupled to said catheter outlet port;
said second inlet port comprising a septum and a second needle entrance opening overlaying said septum;
said first needle entrance opening comprising a slot having a width W₁ greater than 0.0285 inches for passing a 22 gauge needle and a length L₁ greater than four times W₁; and
said second needle entrance opening comprising a slot having a width W₂ greater than 0.0205 inches for passing a 25 gauge needle and less than 0.0275 inches for rejecting a 22 gauge needle and a length L₂ greater than four times W₂.

13. The device of 12 wherein said first inlet port defines a first peripheral wall surface diverging outwardly from said first needle entrance opening slot; and wherein
said second inlet port defines a second peripheral wall surface diverging outwardly from said second needle entrance opening slot.

14. The device of claim 12 wherein said first inlet port defines an oval shaped depression having a sloping peripheral wall converging toward said first needle entrance opening.

15. The device of claim 12 wherein said second inlet port defines an oval shaped depression having a sloping peripheral wall converging toward said second needle entrance opening.

16. A method of configuring a medication delivery device defining an interior volume comprising:
providing a needle receiving port for communicating with said interior volume; and
forming an entrance opening to said port comprising an elongate slot having a length L and a width W where W is selected to prevent penetration by a needle larger than a certain size and where L is considerably greater than W.

17. The method of claim 16 where L is greater than four times W.

18. The method of claim 16 wherein said elongate slot has a width W greater than 0.0205 inches for passing a 25 gauge needle and less than 0.0275 inches for rejecting a 22 gauge needle.

19. The method of claim 16 further including:
forming an oval shaped depression having a sloping peripheral wall converging toward said entrance opening.
